(19) United States
(12) Patent Application Publication
(10) Pub. No.: US 2011/0159479 A1
(43) Pub. Date: Jun. 30, 2011

(54) GENETIC POLYMORPHISMS IN THE CYTOCHROME P450 GENE WITH CLOPIDOGREL RESISTANCE

(75) Inventors: Yang Soo Jang, Seoul (KR); Dongjik Shin, Seoul (KR); Sungha Park, Seoul (KR); Jungmyung Lee, Yongin Si (KR); Chiyoung Shim, Seoul (KR)

(73) Assignee: Industry-University Cooperation Foundation Yonsei University, Seoul (KR)

(21) Appl. No.: 12/648,182

(22) Filed: Dec. 28, 2009

(51) Int. Cl. C12Q 1/68

(52) U.S. Cl. 435/6

(57) ABSTRACT

The present invention relates to a method for predicting the resistance of a human subject to clopidogrel, which comprises detecting the presence or absence of a A allele at position 636 of exon 4 in the CYP2C19 gene, wherein the presence of the A allele is indicative of a clopidogrel resistance. The present method may be very useful in predicting the resistance of a human subject to clopidogrel and contribute to more effective chemotherapy for patients having coronary artery disease and drug-eluting stent.

### BACKGROUND OF THE INVENTION

1. Field of the Invention

The present invention relates to a method and a kit for predicting the resistance of a human subject to clopidogrel.

2. Description of the Related Art

Since clopidogrel and aspirin inhibit platelet aggregation through different pathways, combined antiplatelet therapy provides additive benefits compared to either agent alone,¹ and considered standard therapy in patients undergoing coronary stenting.² However, response variability and nonresponsiveness to clopidogrel have been demonstrated in patients following coronary stenting.³ The prevalence of clopidogrel nonresponsiveness has been reported at 5-44%.⁴⁻¹¹ There are many reports that high platelet reactivity despite clopidogrel therapy is a risk factor of thrombotic event in patients undergoing percutaneous coronary intervention.^{5,12,13} Differences in intestinal absorption, hepatic conversion to the active metabolite through CYP activity, and platelet receptor polymorphisms have been suggested as the mechanism responsible for clopidogrel nonresponsiveness.¹⁴⁻²⁰ Lau et al demonstrated that pharmacological stimulation of CYP3A4 activity enhances the effect of clopidogrel, whereas competitive inhibitor of CYP3A4 attenuate the effect of clopidogrel.^{18,21} The previous studies demonstrated that CYP2C19*2 polymorphism is responsible for clopidogrel resistance.^{14,16,19} These data suggest the contribution of hepatic CYP metabolic activity to clopidogrel nonresponsiveness. However, most of the studies have been done in Caucasians with paucity of data in the Asian populations at the present. In this study, we sought to determine the association of polymorphisms of CYP gene with clopidogrel resistance in subjects undergoing coronary angioplasty and stent insertion.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to reveal genetic background underlying clopidogrel resistance. As a result, we have discovered that a particular genetic polymorphism in the cytochrome P450 gene is closely related to clopidogrel resistance of a patient undergoing coronary angioplasty and stent insertion.

Accordingly, it is an object of this invention to provide a method for predicting the resistance of a human subject to clopidogrel.

It is another object of this invention to provide a kit for predicting the resistance of a human subject to clopidogrel.

Other objects and advantages of the present invention will become apparent from the following detailed description together with the appended claims and drawings.

### DETAILED DESCRIPTION OF THIS INVENTION

In one aspect of this invention, there is provided a method for predicting the resistance of a human subject to clopidogrel, which comprises detecting the presence or absence of a A allele at position 636 of exon 4 in the CYP2C19 gene, wherein the presence of the A allele is indicative of a clopidogrel resistance.

The present inventors have made intensive researches to reveal genetic background underlying clopidogrel resistance. As a result, we have discovered that a particular genetic polymorphism in the cytochrome P450 gene is closely related to clopidogrel resistance of a patient undergoing coronary angioplasty and stent insertion.

The present method may be described as either “a method for predicting the resistance of a human subject to clopidogrel” or “a method for predicting susceptibility to the resistance of a human subject to clopidogrel”.

The present invention is drawn to genetic polymorphisms relating to clopidogrel resistance. Clopidogrel is an oral antiplatelet agent (thienopyridine class) to inhibit blood clots in coronary artery disease, peripheral vascular disease, and cerebrovascular disease. Its IUPAC name is (+)-(S)-methyl 2-(2-chlorophenyl)-2-(6,7-dihydrothieno[3,2-c]pyridin-5(4H)-yl)acetate.

The present invention utilizes a single nucleotide polymorphism in the CYP2C19 (cytochrome P450, family 2, subfamily C, polypeptide 19) gene. The single nucleotide polymorphism is a A nucleotide at position 636 of exon 4 in the CYP2C19 gene. The mRNA sequence of the CYP2C19 gene is described as set forth in SEQ ID NO:1. The A nucleotide variant is a guanine-to-adenine point mutation that produces a premature stop codon “tga” responsible for a change from a tryptophan (W) to a stop codon.

According to a preferred embodiment, the human subject has a patient having coronary artery disease. More preferably, the patient having coronary artery disease has a drug-eluting stent.

According to a preferred embodiment, the human subject is Korean. The term used herein “Korean” refers to a Korean population whose ancestor is also Korean. Preferably, the term “Korean” refers to a Korean population whose at least ten ancestor generations are Korean.

The detection of the A allele at position 636 of exon 4 in the CYP2C19 gene may be carried out by conventional genetic analysis technologies known in the art.

Representative technologies that may be employed include without limitation an amplification reaction (preferably, PCR amplification), a primer extension reaction (Nikiforov, T. T. et al., Nucl Acids Res 22, 4167-4175 (1994)), 5′-exonuclease fluorescence assay (or Taqman assay, U.S. Pat. No. 5,210,015), sunrise primer assay (U.S. Pat. No. 6,117,635), scorpion primer method (U.S. Pat. No. 6,326,145), molecular beacon method (WO 95/13399), a hybridization reaction, a nucleotide sequencing, oligonucleotide ligation analysis (OLA)(Nickerson, D. A. et al., Pro Nat Acad Sci USA, 87, 8923-8927 (1990)), allele-specific PCR (Rust, S. et al., Nucl Acids Res, 6, 3623-3629 (1993)), RNase mismatch cleavage (Myers R. M. et al., Science, 230, 1242-1246 (1985)), single strand conformation polymorphism (SSCP; Orita M. et al., Pro Nat Acad Sci USA, 86, 2766-2770 (1989)), simultaneous analysis of SSCP and heteroduplex (Lee et al., Mol Cells, 5:668-672 (1995)), denaturation gradient gel electrophoresis (DGGE; Cariello N F. et al., Am J Hum Genet, 42, 726-734 (1988)), denaturing high performance liquid chromatography (D-HPLC, Underhill Pa. et al., Genome Res, 7, 996-1005 (1997)), dot blots, MASDA (Multiplexed Allele-Specific Diagnostic Assay), reverse dot blots, ARMS (amplification refractory mutation system), ALEX (arrayed primer extension, EP 332435), COPS (competitive oligonucleotide primer system, Gibbs et al., Nucleic Acids Research, 17:2347 (1989)), APEX (arrayed primer extension), RFLP (restriction fragment length polymorphism) and invader assay (Olivier M, Mutat. Res. 3; 573(1-2):103-10 (2005)).

Some of the genetic analysis technologies use primers.

The term “primer” used herein means an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of four different nucleoside triphosphates and a thermostable enzyme in an appropriate buffer and at a suitable temperature.

The primers having appropriate sequences upstream and downstream of the polymorphic site may be used to amplify the nucleotide regions comprising the polymorphism. Alternatively, the primers specifically hybridized with the A allele at position 636 of exon 4 in the CYP2C19 gene may be designed and used for SNP detection.

The term “probe” used herein refers to a linear oligomer of natural or modified monomers or linkages, including deoxyribonucleotides, ribonucleotides and the like, which is capable of specifically hybridizing with a target nucleotide sequence, whether occurring naturally or produced synthetically. The probe used in the present method may be prepared in the form of oligonucleotide probe, single-stranded DNA probe, double-stranded DNA probe and RNA probe. It may be labeled with biotin, FITC, rhodamine, DIG and radioisotopes.

The probes specifically hybridized with the A allele at position 636 of exon 4 in the CYP2C19 gene may be designed and used for SNP detection.

The nucleic acid samples to be detected may include any type of nucleic acids such as gDNA, cDNA and mRNA.

The amplification reactions using primers may be carried out in accordance with well-known methods. The nucleic acid molecule may be either DNA or RNA. The molecule may be in either a double-stranded or single-stranded form. Where the nucleic acid as starting material is double-stranded, it is preferred to render the two strands into a single-stranded or partially single-stranded form. Methods known to separate strands includes, but not limited to, heating, alkali, formamide, urea and glycoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA is used. The oligonucleotide dT primer is comprised of dTMPs, one or more of which may be replaced with other dNMPs so long as the dT primer can serve as primer. Reverse transcription can be done with reverse transcriptase that has RNase H activity. If one uses an enzyme having RNase H activity, it may be possible to omit a separate RNase H digestion step by carefully choosing the reaction conditions.

The primer used for the present invention is hybridized or annealed to a site on the template such that double-stranded structure is formed. Conditions of nucleic acid annealing suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E. coli DNA polymerase I, a thermostable DNA polymerase, and bacteriophage T7 DNA polymerase. Preferably, the polymerase is a thermostable DNA polymerase which may be obtained from a variety of bacterial species, including Thermus aquaticus (Taq), Thermus thermophilus (Tth), Thermus filiformis, Thermis flavus, Thermococcus literalis, and Pyrococcus furiosus (Pfu). Many of these polymerases may be isolated from bacterium itself or obtained commercially. Polymerase to be used with the subject invention can also be obtained from cells which express high levels of the cloned genes encoding the polymerase.

When a polymerization reaction is being conducted, it is preferable to provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present method is performed under stringent conditions that allow for specific binding between the primer and the template nucleic acid. Such stringent conditions for annealing will be sequence-dependent and varied depending on environmental parameters.

Most preferably, the amplification is performed in accordance with PCR which is disclosed in U.S. Pat. Nos. 4,683,195, 4,683,202, and 4,800,159.

The analysis of amplified products in the present invention may be conducted by various methods or protocols, e.g. electrophoresis such as agarose gel electrophoresis.

Alternatively, the present method may be carried out in accordance with hybridization reaction using suitable probes.

The stringent conditions of nucleic acid hybridization suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985). As used herein the term “stringent condition” refers to the conditions of temperature, ionic strength (buffer concentration), and the presence of other compounds such as organic solvents, under which hybridization or annealing is conducted. As understood by those of skill in the art, the stringent conditions are sequence dependent and are different under different environmental parameters. Longer sequences hybridize or anneal specifically at higher temperatures.

Some modifications in the probes used in this invention can be made unless the modifications abolish the advantages of the oliogonucleotides. Such modifications, i.e., labels linking to the probes generate a signal to detect hybridization. Suitable labels include fluorophores, chromophores, chemiluminescers, magnetic particles, radioisotopes, mass labels, electron dense particles, enzymes, cofactors, substrates for enzymes and haptens having specific binding partners, e.g., an antibody, streptavidin, biotin, digoxigenin and chelating group, but not limited to. The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal.

Preferably, the probes used in the present invention may be immobilized on a solid substrate (nitrocellulose membrane, nylon filter, glass plate, silicon wafer and fluorocarbon support) to fabricate microarray. In microarray, the probes serve as hybridizable array elements.

The probes used in the hybridization reaction have the A allele specific nucleotide sequence.

The present method may be carried out by direct sequencing of gDNA or mRNA. The general processes for sequencing of nucleic acid molecules are found in Sambrook, J. et al., Molecular Cloning. A Laboratory Manual, 3rd ed. Cold Spring Harbor Press (2001), the teachings of which are incorporated herein by reference in their entity.

The nucleic acids to be analyzed may be obtained from various biological samples including tissue, cell, whole blood, serum, plasma, peripheral blood leukocyte, saliva, semen, urine, synovia and spinal fluid.

In another aspect of this invention, there is provided a kit for predicting the resistance of a human subject to clopidogrel, which comprises a primer or a probe hybridizable with a A allele at position 636 of exon 4 in the CYP2C19 gene, wherein the presence of the A allele is indicative of a clopidogrel resistance.

Since the kit of this invention is devised to perform the prediction method of the present invention described above, the common descriptions between them are omitted in order to avoid undue redundancy leading to the complexity of this specification.

The present kits may optionally include the reagents required for performing target amplification PCR reactions (e.g., PCR reactions) such as buffers, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity.

The kits may also include reagents necessary for performing positive and negative control reactions. Optimal amounts of reagents to be used in a given reaction can be readily determined by the skilled artisan having the benefit of the current disclosure. The kits, typically, are adapted to contain in separate packaging or compartments the constituents afore-described.

The present method may be very useful in predicting the resistance of a human subject to clopidogrel and contribute to more effective chemotherapy for patients having coronary artery disease and drug-eluting stent.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Methods

From October 2006 to July 2007, 450 consecutive patients who underwent successful percutaneous coronary intervention (PCI) with drug-eluting stents (DES) were randomly assigned to treatment with dual anti-platelet regimens (aspirin plus clopidogrel, n=225) or triple antiplatelet regimens (aspirin plus clopidogrel plus cilostazol, n=225). Inclusion criteria were: symptomatic coronary artery disease (CAD) or documented myocardial ischemia (by treadmill exercise testing or sestamibi scan); angiographic evidence of 50% diameter stenosis and post procedure Thromobolysis In Myocardial Infarction (TIMI) flow grade 3. Exclusion criteria were: contraindication to antiplatelet agents; previous allergy or intolerance of aspirin or clopidogrel; treatment with warfarin; active bleeding; known platelet dysfunction; abnormal platelet count (<100,000/mm³). Patients received a 300 mg loading dose of clopidogrel at least 12 hours before the stenting. Stents were deployed according to standard techniques. The maintenance dose for each antiplatelet agent was 100 mg once a day for aspirin, 75 mg once a day for clopidogrel, and 100 mg twice a day for cilostazol. Among the enrolled patients, 387 patients were analyzed for clopidogrel resistance by VerifyNow P2Y12 system (Accumetrics, San Diego, Calif.), and blood sampling for genetic analysis.

The VerifyNow P2Y12 system is a rapid platelet-function, cartridge based assay designed to directly measure the effects of clopidogrel on the P2Y12 receptor. Results are expressed as P2Y12 reaction units (PRUs) and percentage inhibition. PRU reports the amount of P2Y12 receptor mediated aggregation. Percent inhibition [(1-PRU/baseline PRU)×100] is the percent change from baseline aggregation and is calculated from the PRU result and the estimated baseline result, which is an independent measurement based on the rate and extent of platelet aggregation in the TRAP (Thrombin Receptor Activating Peptide) channel.²² The VeryfyNow P2Y12 assay's usefulness in evaluating clopidogrel responsiveness is demonstrated in various studies.²²⁻²⁴ The percent inhibition of <20% was defined as clopidogrel resistance.

The genotyping of 7 SNPs including cycloxygenase2 (COX2) (rs5277), CYP1A1 (rs1048943), CYP1A2 (rs2470890), CYP3A4 (rs2242480), CYP3A5 (rs776747), CYP2C19*2 (rs4244285), CYP2C19*3 (rs4986893) were performed using single base primer extension assay using ABI PRISM SNaPshot™ Multiplex kit (Applied Biosystems, Foster City, Calif.) according to manufacturer's recommendation. Briefly, the genomic DNA flanking the SNPs were amplified with polymerase chain reaction (PCR) with forward and reverse primer pairs (CYP1A1: forward primer, GTGATTATCTTTGGCATGG, reverse primer, TTGCAGCAGGATAGCCAG; CYP1A2: forward primer, CGACCTGACCCCCATCTAC, reverse primer, GGAAGAGAAACAAGGGCTGA; CYP2C19*2: forward primer, GGCATATTGTATCTATACCTTTATTAAATG, reverse primer, GAGGGTTGTTGATGTCCATC; CYP2C19*3: forward primer, AGCAATTTCTTAACTTGATGGAAAAA, reverse primer, GGATTTCCCAGAAAAAAAGACTG; CYP3A4: forward primer, CCAGCAGAAACTGCAGG, reverse primer, GAGTCAGTGAAAGAATCAGTGATT; CYP3A5*3: forward primer, CGTTCTGTGTGGGGACAAC, reverse primer, GCCCATACAGGCAACATGA; COX2: forward primer, GCGATTGTACCCGGACAG, reverse primer, TTGGCGATTAAGATGGAAGG) and standard PCR reagents in 10 μl reaction volume, containing 10 ng of genomic DNA, 0.5 pM of each oligonucleotide primer, 1 μl of 10× PCR buffer, 250 μM dNTPs, 3 mM MgCl₂ and 0.25 unit i-StarTaq DNA Polymerase (iNtRON Biotechnology, Sungnam, Korea). The PCR reactions were carried out as follows: 10 min at 95□ for 1 cycle, and 30 cycles on 95□ for 30 sec at 55□ (COX2 rs5277, CYP1A1 rs1048943), at 60□ (CYP2C19*2, CYP2C19*3), at 65□ (CYP3A5*3) for 1 min, respectively, and at 72□ for 1 min followed by 1 cycle of 72□ for 7 min. After amplification, the PCR products were treated with 1 unit each of shrimp alkaline phosphatase (SAP) (Roche, Mannheim, Germany) and exonuclease I (US Biochemical, Cleveland, Ohio) at 37□ for 60 min and 72□ for 15 min to purify the amplified products. 1 μl of the purified amplification products were added to a SNaPShot™ Multiplex Ready reaction mixture containing 0.15 pM of genotyping primer (CYP1A1, AAAGACCTCCCAGCGGGCAA; CYP1A2, CCTCAGAATGGTGGTGTCTTCTTCA; CYP2C19*2, TTTTAAGTAATTTGTTATGGGTTCC; CYP2C19*3, GCAAAAAACTTGGCCTTACCTGGAT; CYP3A4, TACCCAATAAGGTGAGTGGATG; CYP3A5*3, GAGCTCTTTTGTCTTTCA; COX2, TTCGAAATGCAATTATGAGTTATGT) for primer extension reaction. The primer extension reaction was carried out for 25 cycles of 96□ for 10 sec, 50□ for 5 sec, and 60□ for 30 sec. The reaction products were treated with 1 unit of SAP at 37□ for 1 hr and 72□ 15 min to remove excess fluorescent dye terminators. 1 μl of the final reaction samples containing the extension products were added to 9 μl of Hi-Di formamide (Applied Biosystems). The mixture was incubated at 95□ for 5 min, followed by 5 min on ice and then analyzed by electrophoresis in ABI Prism 3730 DNA analyzer. Results were analyzed using GeneScan Analysis Software ver. 3.1 (Applied Biosystems).

The genotyping of CYP3A4 rs2246709, CYP2J2 rs2280274, P2RY12 rs2046934 were screened using the TaqMan fluorogenic 5′ nuclease assay (Applied Biosystems). The final volume of PCR was 5 μl, containing 10 ng of genomic DNA and 2.5 μl TaqMan® Universal PCR master mix, with 0.13 μl of 40× assay mix (Assay ID C_{—}1845287_{—}10 for CYP3A4, C_{—}1917976_{—}1 for CYP2J2, C_{—}1941752_{—}10 for P2RY12). Thermal cycle conditions were as follows: 50□ for 2 min to activate the uracil N-glycosylase and to prevent carry-over contamination, 95□ for 10 min to activate the DNA polymerase, followed by 45 cycles of 95□ for 15 sec and 60□ for 1 min. All PCR were performed using 384-well plates by a Dual 384-Well GeneAmp PCR System 9700 (Applied Biosystems) and the endpoint fluorescent readings were performed on an ABI PRISM 7900 HT Sequence Detection System (Applied Biosystems). Duplicate samples and negative controls were included to ensure accuracy of genotyping.

Values were expressed as mean±SD. Chi-square test for goodness of fit was used to verify agreement with Hardy-Weinberg equilibrium (HWE) using Fisher's exact test. Comparison of discrete variables was performed using the Chi-square test analysis or Fisher's exact test. Comparison of continuous variables between the two study groups was performed using the Student's t-test. Multivariate logistic regression analysis was performed to determine the independent association of CYP gene polymorphisms with clopidogrel resistance. Odds ratios (ORs) were calculated with 95% confidence intervals (CIs) for the relative risk of clopidogrel resistance related to genotypes. Statistical analysis was performed with SPSS 15.0 (SPSS Inc, Chicago, Ill.). All values of P<0.05 were considered statistically significant.

### Results

CYP genotypes and clopidogrel resistance of 387 CAD patients with DES insertion were analyzed in this study. Clopidogrel resistance was found in 112 patients (28.9%). The population was divided into two groups according to the presence of clopidogrel resistance assessed by VerifyNow P2Y12 assay. No significant differences in age, sex, body mass index (BMI), history of diabetes mellitus, history of hypertension, and smoking status were seen between clopidogrel resistant group and clopidogrel responsive group. In clopidogrel responsive group, there was a significant higher proportion of cilostazol use (Table 1). It is a consistent finding with previous report that addition of cilostazol to conventional dual antiplatelet regimen can attenuate clopidogrel resistance.²⁵

TABLE 1
Clinical characteristics of clopidogrel
resistant and responsive groups

| Characteristics | No resistance | Resistance | P value |
|---|---|---|---|
| Age (years) | 61.1 ± 10.3 | 61.5 ± 9.7 | 0.719 |
| Body mass index (kg/m²) | 24.7 ± 2.9 | 25.0 ± 3.1 | 0.538 |
| Men | 199 (72.4%) | 82 (73.2%) | 0.865 |
| Diabetes mellitus | 80 (30.2%) | 25 (25.9%) | 0.399 |
| Hypertension | 121 (44.0%) | 46 (41.1%) | 0.598 |
| Smoker | 110 (40.0%) | 32 (28.6%) | 0.034 |
| Cilostazol | 158 (57.5%) | 32 (28.6%) | 0.0000003 |

Values are expressed as mean ± SD or as percentages.

The distribution of the genetic polymorphisms in clopidogrel responsive group did not deviate significantly from the HWE, except for CYP2C19*2 (Table 2). Repetition of genotyping was done and did not find genotyping error. Genotyping call rates for all SNPs ranged from 97.2% to 100% (Table 2).

TABLE 2
Hardy-Weinberg equilibrium of studied polymorphisms

| | | Frequency | | Call | HWE | | |
|---|---|---|---|---|---|---|---|
| Gene | SNP | Major | Minor | rate, % | Total | No resistance | Resistance |
| CYP1A1 | rs1048943 | 575 | 193 | 99.2 | 0.2802 | 0.2135 | 1 |
| CYP1A2 | rs2470890 | 641 | 133 | 100.0 | 0.5983 | 0.5017 | 1 |
| CYP2C19 | rs4244285 | 553 | 211 | 98.7 | 0.0167 | 0.0422 | 0.1819 |
| CYP2C19 | rs4986893 | 700 | 72 | 99.7 | 0.5607 | 1 | 0.4529 |
| CYP3A4 | rs2246709 | 341 | 236 | 98.4 | 0.0334 | 0.0645 | 0.3063 |
| CYP3A4 | rs2242480 | 614 | 160 | 100.0 | 0.4403 | 0.7261 | 0.3627 |
| CYP3A5 | rs776746 | 573 | 179 | 97.2 | 0.3945 | 0.4099 | 1 |
| CYP2J2 | rs2280274 | 377 | 74 | 98.4 | 1 | 0.7575 | 0.2776 |
| P2RY12 | rs2046934 | 631 | 139 | 99.5 | 1 | 0.5588 | 0.2800 |
| COX2 | rs5277 | 743 | 31 | 100.0 | 1 | 1 | 1 |

SNP = single nucleotide polymorphism; CYP = cytochrome P450; COX2 = cyclo-oxygenase2; HWE = Hardy-Weinberg equilibrium.

Genetic distributions of the 10 SNPs are shown in Table 3. Among the 10 SNPs, the frequency of CYP2C19*3 A allele was significantly higher in the clopidogrel resistant group than in responsive group (GG:GA:AA=235:35:1 vs. 79:31:1, respectively, P=0.001).

TABLE 3
Genetic polymorphisms distribution in clopidogrel resistant and responsive groups

| | | | | No resistance | Resistance | |
|---|---|---|---|---|---|---|
| Gene | SNP | | Genotype | (n = 275) | (n = 112) | P value |
| CYP1A1 | rs1048943 | Codominant | AA | 143 | 68 | 0.096 |
| | | | AG | 116 | 37 | |
| | | | GG | 15 | 5 | |
| | | Dominant | AA | 143 | 68 | 0.09 |
| | | | AG/GG | 131 | 42 | |
| | | Recessive | AA/AG | 259 | 105 | 0.805 |
| | | | GG | 15 | 5 | |
| CYP1A2 | rs2470890 | Codominant | CC | 194 | 73 | 0.312 |
| | | | CT | 72 | 35 | |
| | | | TT | 9 | 4 | |
| | | Dominant | CC | 194 | 73 | 0.333 |
| | | | CT/TT | 81 | 39 | |
| | | Recessive | CC/CT | 266 | 108 | 1 |
| | | | TT | 9 | 4 | |
| CYP2C19*2 | rs4244285 | Codominant | GG | 155 | 55 | 0.287 |
| | | | GA | 93 | 40 | |
| | | | AA | 26 | 13 | |
| | | Dominant | GG | 155 | 55 | 0.361 |
| | | | GA/AA | 119 | 53 | |
| | | Recessive | GG/GA | 248 | 95 | 0.457 |
| | | | AA | 26 | 13 | |
| CYP2C19*3 | rs4986893 | Codominant | GG | 236 | 80 | 0.001 |
| | | | GA | 37 | 31 | |
| | | | AA | 1 | 1 | |
| | | Dominant | GG | 236 | 80 | 0.001 |
| | | | GA/AA | 38 | 32 | |
| | | Recessive | GG/GA | 273 | 111 | 0.497 |
| | | | AA | 1 | 1 | |
| CYP3A4 | rs2246709 | Codominant | TT | 103 | 42 | 0.925 |
| | | | TC | 139 | 57 | |
| | | | CC | 28 | 12 | |
| | | Dominant | TT | 103 | 42 | 1 |
| | | | TC/CC | 167 | 69 | |
| | | Recessive | TT/TC | 242 | 99 | 0.857 |
| | | | CC | 28 | 12 | |
| CYP3A4 | RS2242480 | Codominant | GG | 172 | 74 | 0.568 |
| | | | GA | 90 | 32 | |
| | | | AA | 13 | 6 | |
| | | Dominant | GG | 172 | 74 | 0.561 |
| | | | GA/AA | 103 | 38 | |
| | | Recessive | GG/GA | 262 | 106 | 0.798 |
| | | | AA | 13 | 6 | |
| CYP3A5 | rs776746 | Codominant | GG | 154 | 61 | 0.808 |
| | | | GA | 102 | 41 | |
| | | | AA | 12 | 6 | |
| | | Dominant | GG | 154 | 61 | 0.908 |
| | | | GA/AA | 114 | 47 | |
| | | Recessive | GG/GA | 256 | 102 | 0.606 |
| | | | AA | 12 | 6 | |
| CYP2J2 | rs2280274 | Codominant | TT | 216 | 91 | 0.695 |
| | | | TA | 52 | 18 | |
| | | | AA | 2 | 2 | |
| | | Dominant | TT | 216 | 91 | 0.776 |
| | | | TA/AA | 54 | 20 | |
| | | Recessive | TT/TA | 268 | 109 | 0.583 |
| | | | AA | 2 | 2 | |
| P2RY12 | rs2046934 | Codominant | TT | 177 | 81 | 0.139 |
| | | | TC | 89 | 26 | |
| | | | CC | 8 | 4 | |
| | | Dominant | TT | 177 | 81 | 0.121 |
| | | | TC/CC | 97 | 30 | |
| | | Recessive | TT/TC | 266 | 107 | 0.75 |
| | | | CC | 8 | 4 | |
| COX2 | rs5277 | Codominant | GG | 253 | 103 | 0.991 |
| | | | GC | 22 | 9 | |
| | | | CC | 0 | 0 | |
| | | Dominant | GG | 253 | 103 | 0.991 |
| | | | GC/CC | 22 | 9 | |

SNP = single nucleotide polymorphism; CYP = cytochrome P450; COX2 = cyclo-oxygenase2.

Table 4 shows the comparison of percent inhibition according to each genotype (non-significant data not shown). Dominant model of CYP1A1 rs1048943, and CYP2C19*2 polymorphism indicated a significant difference in percent inhibition. CYP19*3 demonstrated significantly different percent inhibition in both codominant and dominant model.

TABLE 4
Percent inhibition of platelet activity according to genotypes

| Gene | SNP | | Genotype (n) | % inhibition | P value |
|---|---|---|---|---|---|
| CYP1A1 | rs1048943 | Codominant | AA (211) | 32.5 ± 22.3 | 0.081 |
| | | | AG (153) | 37.7 ± 24.2 | |
| | | | GG (20) | 38.7 ± 24.4 | |
| | | Dominant | AA (210) | 32.5 ± 22.3 | 0.029 |
| | | | AG/GG (170) | 37.8 ± 24.2 | |
| | | Recessive | AA/AG (361) | 34.7 ± 23.2 | 0.450 |
| | | | GG (19) | 38.7 ± 24.4 | |
| CYP2C19*2 | rs4244285 | Codominant | GG (210) | 37.2 ± 24.5 | 0.100 |
| | | | GA (133) | 32.5 ± 21.6 | |
| | | | AA (39) | 30.9 ± 21.7 | |
| | | Dominant | GG (210) | 37.2 ± 24.5 | 0.048 |
| | | | GA/AA (172) | 32.1 ± 21.6 | |
| | | Recessive | GG/GA (343) | 35.3 ± 23.5 | 0.256 |
| | | | AA (39) | 30.9 ± 21.7 | |
| CYP2C19*3 | rs4986893 | Codominant | GG (316) | 36.9 ± 23.4 | 0.0003 |
| | | | GA (68) | 24.9 ± 20.1 | |
| | | | AA (2) | 17.0 ± 24.0 | |
| | | Dominant | GG (316) | 36.9 ± 23.4 | 0.00004 |
| | | | GA/AA (70) | 24.6 ± 20.0 | |
| | | Recessive | GG/GA (384) | 34.8 ± 23.3 | 0.287 |
| | | | AA (2) | 17.0 ± 24.0 | |

CYP = cytochrome P450; SNP = single nucleotide polymorphism.

Because cilostazol influent clopidogrel resistance significantly, we examined the association of SNPs and clopidogrel resistance in dual antiplatelet therapy group and triple antiplatelet group, respectively. In the dual and triple antiplatelet therapy population, CYP2C19*3 A allele was significantly more prevalent in clopidogrel resistant group than responsive group (P=0.01 and P=0.003, respectively, data not shown). No significant associations between any other SNPs and clopidogrel resistance were seen.

Finally, multiple logistic regression analysis was performed for the 10 SNPs after adjustment for age, sex, history of diabetes, smoking status, cilostazol use, and BMI. The results demonstrated that CYP2C19*3 polymorphism is an independent predictor of clopidogrel resistance (Table 5). Cilostazol attenuates clopidogrel resistance significantly as previously reported (OR 0.397, 95% CI: 0.246-0.640; P=0.000001).

TABLE 5
Associations of studied genetic polymorphisms with clopidogrel resistance

| | Dominant | | Codominant | | Recessive | |
|---|---|---|---|---|---|---|
| | OR (95% CI) | P value | OR (95% CI) | P value | OR (95% CI) | P value |
| CYP1A1 rs1048943* | 0.680 (0.421-1.098) | 0.114 | 0.717 (0.475-1.081) | 0.112 | 0.655 (0.204-2.101) | 0.477 |
| CYP1A2 rs2470890* | 1.302 (0.792-2.140) | 0.298 | 1.269 (0.828-1.945) | 0.275 | 1.468 (0.407-5.298) | 0.558 |
| CYP2C19*2 rs4244285* | 1.338 (0.834-2.147) | 0.227 | 1.294 (0.915-1.831) | 0.145 | 1.612 (0.761-3.417) | 0.212 |
| CYP2C19*3 rs4986893* | 2.639 (1.486-4.686) | 0.001 | 2.613 (1.510-4.522) | 0.001 | 7.793 (0.447-135.832) | 0.159 |
| CYP3A4 rs2246709* | 0.965 (0.595-1.566) | 0.885 | 1.011 (0.700-1.460) | 0.953 | 1.142 (0.541-2.413) | 0.727 |
| CYP3A4 rs2242480* | 0.850 (0.524-1.381) | 0.512 | 0.920 (0.616-1.374) | 0.683 | 1.213 (0.426-3.454) | 0.718 |
| CYP3A5 rs776746* | 1.002 (0.621-1.616) | 0.994 | 1.014 (0.682-1.507) | 0.946 | 1.092 (0.381-3.129) | 0.870 |
| CYP2J2 rs2280274* | 0.919 (0.499-1.692) | 0.786 | 1.013 (0.580-1.768) | 0.964 | 3.490 (0.449-27.115) | 0.232 |
| P2RY12 rs2046934* | 0.637 (0.381-1.066) | 0.086 | 0.685 (0.435-1.077) | 0.102 | 0.721 (0.176-2.954) | 0.649 |
| COX rs5277* | 0.883 (0.366-2.134) | 0.783 | 0.883 (0.366-2.134) | 0.783 | — | — |
| Cilostazol^{§} | 3.474 (0.246-0.640) | 0.000001 | — | — | — | — |

*Adjusted for age, sex, history of diabetes, smoking status, cilostazol use and BMI.
^{§}Adjusted for age, sex, history of diabetes, smoking status, BMI, and CYP2C19*3 dominant.
COX2 = cyclo-oxygenase2; CYP = cytochrome P450; SNP = single nucleotide polymorphism.

### Discussion

The primary finding from this study was that CYP2C19*3 polymorphism significantly affected clopidogrel resistance in patients with coronary disease treated with coronary angioplasty and DES implantation. We found that CYP2C19*3 A allele carriers had a higher proportion of clopidogrel resistance. The association remained significant after adjustment for other clinical factors such as age, sex, BMI, smoking status, and history of diabetes. Multiple logistic regression demonstrated that it is an independent predictor of clopidogrel resistance. CYP2C19*3 may do more important role in activity of CYP than any other surveyed variants in Koreans. Because active metabolite of clopidogrel arises from biochemical reactions involving several CYP isoforms, one CYP gene variant may not explain all the variability of clopidogrel response. Savi et al²⁶ demonstrated that CYP1A activity plays key role in clopidogrel metabolism, while Lau and Gurbel¹⁸ found that CYP3A4 activity was associated with variability in clopidogrel responsiveness. Hulot et al reported that CYP2C19*2 polymorphism is associated with clopidogrel resistance which has since been validated in several other studies as well.¹⁴⁻¹⁶

De Morais et al²⁷ was first to describe CYP2C19*3 (previous designated CYP2C19ₘ₂) variant in the Japanese population. This variant is a G to A point mutation at position 636 of exon 4 in CYP2C19 gene that produces a premature stop codon.²⁸ According to the original report, there were marked interracial differences in the frequency of the CYP2C19*3, with 9 of 34 alleles being detected in Japanese poor metabolizers. However, it was not detected in Caucasians poor metabolizers. Because of the paucity of CYP2C19*3 A allele in Caucasians, its role was incompletely studied. Hulot et al reported that lack of CYP2C19*3 A allele in young healthy whites, although sample size was too small that could not be able to stand for Caucasians.¹⁹ Moreover, another study presented that the occurrence of CYP2C19*3 A allele is less than 1% in whites.²⁸ Our results imply, for the first time, that CYP2C19*3 is a significant risk factor for clopidogrel resistance, and may have significant importance in clopidogrel metabolism in the Asian populations. Further investigations are required to elucidate the functional influence of CYP2C19*3 on the response to clopidogrel loading dose. The activation of P2Y12 receptor by ADP results in inhibition of adenylyl cyclase and decreases in cyclic adenosine monophosphate (cAMP) level. The decrease of cAMP results in diminished phosphorylation of vasodilator stimulated phosphoprotein (VASP), resulting in decreased inhibition of GPIIb/IIIa receptor activation. The administration of cilostazole, by increasing cAMP, have been demonstrated in clinical studies to attenuate the degree of plavix resistance.^{28,29} It is interesting to note that although the addition of cilostazol significantly reduces the rate of clopidogrel predictor of clopidogrel resistance in subjects administered with a triple regimen of antiplatelet. This demonstrates that adding another antiplatelet agent is not enough to completely overcome the adverse effect of CYP2C19*3 polymorphism on clopidogrel metabolism. However, the subjects with CYP2C19*3 A allele administered with triple regimen therapy had a much lower rate of clopidogrel resistance (37.1%) compared to subjects with CYP2C19*3 A allele who were administered with dual regimen therapy (57.6%, data not shown). Although speculative at this time, an addition of drugs to increase the activity of the CYP2C19 activity may be one way to enhance the potency of clopidogrel and overcome clopidogrel resistance. Also, further investigation is needed to clarify whether the increased clopidogrel resistance in subjects with CYP2C19*3 A allele translates into increased cardiovascular outcomes.

Having described a preferred embodiment of the present invention, it is to be understood that variants and modifications thereof falling within the spirit of the invention may become apparent to those skilled in this art, and the scope of this invention is to be determined by appended claims and their equivalents.

### ACKNOWLEDGEMENT

This work was supported by a grant from Ministry of Health and Welfare, Republic of Korea (A000385).

### REFERENCES

1. Gurbel P A, Tantry U S. Clopidogrel resistance? Thromb Res 2007; 120:311-321.
2. Steinhubl S R, Berger P B, Mann J T, Fry E T, DeLago A, Wilmer C, Topol E J. Early and sustained dual oral antiplatelet therapy following percutaneous coronary intervention: a randomized controlled trial. JAMA 2002; 288:2411-2420.
3. Gurbel P A, Bliden K P, Hayes K M, Yoho J A, Herzog W R, Tantry U S. The relation of dosing to clopidogrel responsiveness and the incidence of high post-treatment platelet aggregation in patients undergoing coronary stenting. J Am Coll Cardiol 2005; 45:1392-1396.
4. Angiolillo D J, Fernandez-Ortiz A, Bernardo E, Ramirez C, Barrera-Ramirez C, Sabate M, Hernandez R, Moreno R, Escaned J, Alfonso F, Banuelos C, Costa M A, Bass T A, Macaya C. Identification of low responders to a 300-mg clopidogrel loading dose in patients undergoing coronary stenting. Thromb Res 2005; 115:101-108.
5. Matetzky S, Shenkman B, Guetta V, Shechter M, Bienart R, Goldenberg I, Novikov I, Pres H, Savion N, Varon D, Hod H. Clopidogrel resistance is associated with increased risk of recurrent atherothrombotic events in patients with acute myocardial infarction. Circulation 2004; 109:3171-3175.
6. Mobley J E, Bresee S J, Wortham D C, Craft R M, Snider C C, Carroll R C. Frequency of nonresponse antiplatelet activity of clopidogrel during pretreatment for cardiac catheterization. Am J Cardiol 2004; 93:456-458.
7. Muller I, Besta F, Schulz C, Massberg S, Schonig A, Gawaz M. Prevalence of clopidogrel non-responders among patients with stable angina pectoris scheduled for elective coronary stent placement. Thromb Res 2003; 89:783-787.
8. Angiolillo D J, Fernandez-Ortiz A, Bernardo E, Ramirez C, Sabate M, Jimenez-Quevedo P, Hernandez R, Moreno R, Escaned J, Alfonso F, Banuelos C, Costa M A, Bass T A, Macaya C. Platelet function profiles in patients with type 2 diabetes and coronary artery disease on combined aspirin and clopidogrel treatment. Diabetes 2005; 54:2430-2435.
9. Angiolillo D J, Fernandez-Ortiz A, Bernardo E, Ramírez C, Barrera-Ramirez C, Sabaté M, Hernández R, Moreno R, Escaned J, Alfonso F, Bañuelos C, Costa M A, Bass T A, Macaya C. Identification of low responders to a 300-mg clopidogrel loading dose in patients undergoing coronary stenting. Thromb Res 2005; 115:101-108.
10. Järemo P, Lindahl T L, Fransson S G, Richter A. Individual variations of platelet inhibition after loading doses of clopidogrel. J Intern Med 2002; 252:233-238.
11. Müller I, Besta F, Schulz C, Massberg S, Schönig A, Gawaz M. Prevalence of clopidogrel non-responders among patients with stable angina pectoris scheduled for elective coronary stent placement. Thromb Haemost 2003; 89:783-787.
12. Gurbel P A, Bliden K P, Tantry U S. Effect of clopidogrel with and without eptifibatide on tumor necrosis factor-alpha and C-reactive protein release after elective stenting: results from the CLEAR PLATELETS 1b study. J Am Coll Cardiol 2006; 48:2186-2191.
13. Gurbel P A, Bliden K P, Zaman K A, Yoho J A, Hayes K M, Tantry U S. Clopidogrel loading with eptifibatide to arrest the reactivity of platelets: results of the Clopidogrel Loading With Eptifibatide to Arrest the Reactivity of Platelets (CLEAR PLATELETS) study. Circulation 2005; 111:1153-1159.
14. Frere C, Cuisset T, Morange P E, Quilici J, Camoin-Jau L, Saut N, Fulle D, Lambert M, Juhan-Vague I, Bonnet J L, Alessi M C. Effect of Cytochrome P450 Polymorphisms on Platelet Reactivity After Treatment With Clopidogrel in Acute Coronary Syndrome. Am J Cardiol 2008; 101:1088-1093.
15. Fontana P, Senouf D, Mach F. Biological effect of increased maintenance dose of clopidogrel in cardiovascular outpatients and influence of the cytochrome P450 2C19*2 allele on clopidogrel responsiveness. Thromb Res 2008; 121:463-468.
16. Giusti B, Gori A M, Marcucci R, Saracini C, Sestini I, Paniccia R, Valente S, Antoniucci D, Abbate R, Gensini G F. Cytochrome P450 2C19 loss-of-function polymorphism, but not CYP3A4 IVS10+12G/A and P2Y12 T744C polymorphisms, is associated with response variability to dual antiplatelet treatment in high-risk vascular patients. Pharmacogenet Genomics 2007; 17:1057-1064.
17. Suh J W, Koo B K, Zhang S Y, Park K W, Cho J Y, Jang I J, Lee D S, Sohn D W, Lee M M, Kim H S. Increased risk of atherothrombotic events associated with cytochrome P450 3A5 polymorphism in patients taking clopidogrel. CMAJ 2006; 174:1715-1722.
18. Lau W C, Gurbel P A. Antiplatelet drug resistance and drug-drug interactions: Role of cytochrome P450 3A4. Pharm Res 2006; 23:2691-2708.
19. Hulot J S, Bura A, Villard E, Azizi M, Remones V, Goyenvalle C, Aiach M, Lechat P, Gaussem P. Cytochrome P450 2C19 loss-of-function polymorphism is a major determinant of clopidogrel responsiveness in healthy subjects. Blood 2006; 108:2244-2247.
20. Angiolillo D J, Fernandez-Ortiz A, Bernardo E, Ramírez C, Cavallari U, Trabetti E, Sabaté M, Hernández R, Moreno R, Escaned J, Alfonso F, Bañuelos C, Costa M A, Bass T A, Pignatti P F, Macaya C. Contribution of gene sequence variations of the hepatic cytochrome P450 3A4 enzyme to variability in individual responsiveness to clopidogrel. Arterioscler Thromb Vasc Biol 2006; 26:1895-1900.
21. Lau W C, Waskell L A, Watkins P B, Neer C J, Horowitz K, Hopp A S, Tait A R, Carville D G, Guyer K E, Bates E R. Atorvastatin reduces the ability of clopidogrel to inhibit platelet aggregation: a new drug-drug interaction. Circulation 2003; 107:32-37.
22. Malinin A, Pokov A, Swaim L, Kotob M, Serebruany V. Validation of a VerifyNow-P2Y12 cartridge for monitoring platelet inhibition with clopidogrel. Methods Find Exp Clin Pharmacol 2006; 28:315-322.
23. Jakubowski J A, Payne C D, Li Y G, Brandt J T, Small D S, Farid N A, Salazar D E, Winters K J. The use of the VerifyNow P2Y12 point-of-care device to monitor platelet function across a range of P2Y12 inhibition levels following prasugrel and clopidogrel administration. Thromb Haemost 2008; 99:409-415.
24. Malinin A, Pokov A, Spergling M, Defranco A, Schwartz K, Schwartz D, Mahmud E, Atar D, Serebruany V. Monitoring platelet inhibition after clopidogrel with the VerifyNow-P2Y12(R) rapid analyzer: the VERIfy Thrombosis risk ASsessment (VERITAS) study. Thromb Res 2007; 119:277-284.
25. Lee S W, Park S W, Kim Y H, Yun S C, Park D W, Lee C W, Hong M K, Kim H S, Ko J K, Park J H, Lee J H, Choi S W, Seong I W, Cho Y H, Lee N H, Kim J H, Chun K J, Park S J. Drug-eluting stenting followed by cilostazol treatment reduces late restenosis in patients with diabetes mellitus the DECLARE-DIABETES Trial (A Randomized Comparison of Triple Antiplatelet Therapy with Dual Antiplatelet Therapy After Drug-Eluting Stent Implantation in Diabetic Patients). J Am Coll Cardiol 2008; 51:1181-1187.
26. Savi P, Combalbert J, Gaich C, Rouchon M C, Maffrand J P, Berger Y, Herbert J M. The antiaggregating activity of clopidogrel is due to a metabolic activation by the hepatic cytochrome P450-1A. Thromb Haemost 1994; 72:313-317.
27. De Morais S M, Wilkinson G R, Blaisdell J, Meyer U A, Nakamura K, Goldstein J A. Identification of a new genetic defect responsible for the polymorphism of (S)-mephenytoin metabolism in Japanese. Mol Pharmacol 1994; 46:594-598.
28. Xie H G, Kim R B, Wood A J, Stein C M. Molecular basis of ethnic differences in drug disposition and response. Annu Rev Pharmacol Toxicol 2001; 41:815-850.
29. Angiolillo D J, Fernandez-Ortiz A, Bernardo E, Alfonso F, Macaya C, Bass T A, Costa M A. Variability in individual responsiveness to clopidogrel: clinical implications, management and future perspectives. J Am Coll Cardiol 2007; 49:1505-1516.

What is claimed is:

1. A method for predicting the resistance of a human subject to clopidogrel, which comprises detecting the presence or absence of a A allele at position 636 of exon 4 in the CYP2C19 gene, wherein the presence of the A allele is indicative of a clopidogrel resistance.

2. The method according to claim 1, wherein the human subject has a patient having coronary artery disease.

3. The method according to claim 2, wherein the patient having coronary artery disease has a drug-eluting stent.

4. The method according to claim 1, wherein the detection is carried out by an amplification reaction, a primer extension reaction, 5′-exonuclease fluorescence assay, a hybridization reaction or a nucleotide sequencing.

5. A kit for predicting the resistance of a human subject to clopidogrel, which comprises a primer or a probe hybridizable with a A allele at position 636 of exon 4 in the CYP2C19 gene, wherein the presence of the A allele is indicative of a clopidogrel resistance.

6. The kit according to claim 5, wherein the human subject has a patient having coronary artery disease.

7. The kit according to claim 6, wherein the patient having coronary artery disease has a drug-eluting stent.


SEQUENCE LISTING (22 sequences)

SEQ ID NO: 1 - DNA, 1473 bp, Homo sapiens
atggatccttttgtggtccttgtgctctgtctctcatgtttgcttctcctttcaatctggagacagagctctgggagaggaaaactccctcctggccccactcctctcccagtgattggaaatatcctacagatagatattaaggatgtcagcaaatccttaaccaatctctcaaaaatctatggccctgtgttcactctgtattttggcctggaacgcatggtggtgctgcatggatatgaagtggtgaaggaagccctgattgatcttggagaggagttttctggaagaggccatttcccactggctgaaagagctaacagaggatttggaatcgttttcagcaatggaaagagatggaaggagatccggcgtttctccctcatgacgctgcggaattttgggatggggaagaggagcattgaggaccgtgttcaagaggaagcccgctgccttgtggaggagttgagaaaaaccaaggcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctgaatccagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggaaaagcaaaaccaacagtctgaattcactattgaaaacttggtaatcactgcagctgacttacttggagctgggacagagacaacaagcacaaccctgagatatgctctccttctcctgctgaagcacccagaggtcacagctaaagtccaggaagagattgaacgtgtcattggcagaaaccggagcccctgcatgcaggacaggggccacatgccctacacagatgctgtggtgcacgaggtccagagatacatcgacctcatccccaccagcctgccccatgcagtgacctgtgacgttaaattcagaaactacctcattcccaagggcacaaccatattaacttccctcacttctgtgctacatgacaacaaagaatttcccaacccagagatgtttgaccctcgtcactttctggatgaaggtggaaattttaagaaaagtaactacttcatgcctttctcagcaggaaaacggatttgtgtgggagagggcctggcccgcatggagctgtttttattcctgaccttcattttacagaactttaacctgaaatctctgattgacccaaaggaccttgacacaactcctgttgtcaatggatttgcttctgtcccgcccttctatcagctgtgcttcattcctgtctga

SEQ ID NO: 2 - DNA, 19 bp, Artificial Sequence
  primer
gtgattatctttggcatgg

SEQ ID NO: 3 - DNA, 18 bp, Artificial Sequence
  primer
ttgcagcaggatagccag

SEQ ID NO: 4 - DNA, 19 bp, Artificial Sequence
  primer
cgacctgacccccatctac

SEQ ID NO: 5 - DNA, 20 bp, Artificial Sequence
  primer
ggaagagaaacaagggctga

SEQ ID NO: 6 - DNA, 30 bp, Artificial Sequence
  primer
ggcatattgtatctatacctttattaaatg

SEQ ID NO: 7 - DNA, 20 bp, Artificial Sequence
  primer
gagggttgttgatgtccatc

SEQ ID NO: 8 - DNA, 26 bp, Artificial Sequence
  primer
agcaatttcttaacttgatggaaaaa

SEQ ID NO: 9 - DNA, 23 bp, Artificial Sequence
  primer
ggatttcccagaaaaaaagactg

SEQ ID NO: 10 - DNA, 17 bp, Artificial Sequence
  primer
ccagcagaaactgcagg

SEQ ID NO: 11 - DNA, 24 bp, Artificial Sequence
  primer
gagtcagtgaaagaatcagtgatt

SEQ ID NO: 12 - DNA, 19 bp, Artificial Sequence
  primer
cgttctgtgtggggacaac

SEQ ID NO: 13 - DNA, 19 bp, Artificial Sequence
  primer
gcccatacaggcaacatga

SEQ ID NO: 14 - DNA, 18 bp, Artificial Sequence
  primer
gcgattgtacccggacag

SEQ ID NO: 15 - DNA, 20 bp, Artificial Sequence
  primer
ttggcgattaagatggaagg

SEQ ID NO: 16 - DNA, 20 bp, Artificial Sequence
  primer
aaagacctcccagcgggcaa

SEQ ID NO: 17 - DNA, 25 bp, Artificial Sequence
  primer
cctcagaatggtggtgtcttcttca

SEQ ID NO: 18 - DNA, 25 bp, Artificial Sequence
  primer
ttttaagtaatttgttatgggttcc

SEQ ID NO: 19 - DNA, 25 bp, Artificial Sequence
  primer
gcaaaaaacttggccttacctggat

SEQ ID NO: 20 - DNA, 22 bp, Artificial Sequence
  primer
tacccaataaggtgagtggatg

SEQ ID NO: 21 - DNA, 18 bp, Artificial Sequence
  primer
gagctcttttgtctttca

SEQ ID NO: 22 - DNA, 25 bp, Artificial Sequence
  primer
ttcgaaatgcaattatgagttatgt